(19) United States
(12) Patent Application Publication
(10) Pub. No.: US 2007/0099245 A1
(43) Pub. Date: May 3, 2007

(54) ASSAYS FOR NEUTRALIZING ANTIBODIES

(76) Inventors: Boris Gorovits, Nanuet, NY (US); John Nowak, Stratham, NH (US); Denise O'Hara, Reading, MA (US); William M. Dickerson, Dorchester, MA (US); Tony Celeste, Medford, MA (US)

Correspondence Address: WYETH/FINNEGAN HENDERSON, LLP, 901 NEW YORK AVENUE, NW, WASHINGTON, DC, 20001-4413, US

(21) Appl. No.: 11/537,178

(22) Filed: Sep. 29, 2006

Related U.S. Application Data
(60) Provisional application No. 60/721,586, filed on Sep. 29, 2005.

(51) Int. Cl. G01N 33/567; G01N 33/53

(52) U.S. Cl. 435/7.2

(57) ABSTRACT

This invention relates to assays for the detection of neutralizing antibodies. Further, this invention relates to assays for the detection of neutralizing antibodies specific for bone morphogenetic protein 2 (BMP-2) or capable of inhibiting at least one of the biological activities of BMP-2.

### PRIOR APPLICATIONS

This application claims priority to U.S. Provisional Application No. 60/721,586, filed on Sep. 29, 2005, the contents of which are hereby incorporated by reference.

### FIELD OF THE INVENTION

This invention relates to assays for the detection of neutralizing antibodies of bone morphogenetic proteins (BMPs). Certain embodiments of this invention include assays for the detection of neutralizing antibodies specific for bone morphogenetic protein 2 (BMP-2).

### BACKGROUND OF THE INVENTION

Advances in biotechnology have made it possible to produce a wide variety of protein therapeutics, including recombinant growth factors. These protein therapeutics pose unique challenges not typically encountered with chemical pharmaceutical compounds. For example, proteins are often less stable than chemical compounds and require special preparation or storage to preserve their activity. In addition, the administration of a protein therapeutic to either animals or humans may elicit an immune response in the subject, because the protein may be recognized as a foreign substance by the subject's immune system. Cell-mediated immune responses and the production of antibodies specific for the protein may result.

One particular protein therapeutic in use today is recombinant human BMP-2 (rhBMP-2). This protein may be used, for example, in combination with an absorbable collagen sponge for spinal fusion and fracture repair. Other BMPs are anticipated to be used for similar indications and for repair of various other tissues. The potential consequences of circulating antibodies specific for a BMP are a serious clinical concern. Because BMPs have natural counterparts in the body, the antibodies may react with the endogenous BMP. And due to the high levels of homology between members of the BMP protein family, the circulating antibodies may cross-react with related endogenous BMP molecules leading to aberrant immune responses, inflammation or allergic reactions. Further, repeated drug administrations may lead to an increased antibody response over time. These concerns are heightened when the patient is pregnant or in women of child-bearing potential because the circulating antibodies may pose additional risks to the reproductive system or a developing fetus.

Neutralizing antibodies, i.e., antibodies that interfere with the biological properties of the BMP, may also be generated upon the administration of the BMP to a subject. In addition to the concerns above, neutralizing antibodies can impact the efficacy or availability of the BMP therapeutic. For example, the antibodies may interfere with the biological activity of the BMP, necessitating a dosage increase to achieve the desired clinical result. Neutralizing antibodies may also alter the rate of drug clearance in either a positive or negative manner.

Neutralizing antibody titers must be monitored in patients taking a BMP therapeutic to ensure safety and efficacy. Consequently, physicians require tests that allow for the rapid and routine monitoring of neutralizing antibodies in their patients so that the administration of the BMP therapeutic may be increased, decreased, or stopped altogether, depending on the test results. In view of the clinical importance of monitoring a patient's immune response to a protein therapeutic, there exists a need to develop assays that allow the detection of neutralizing antibodies specific for the therapeutic protein.

### SUMMARY OF THE INVENTION

The invention provides an assay for the detection of neutralizing antibodies and non-antibody inhibitors of a protein therapeutic.

In certain embodiments, the assays are used to detect neutralizing antibodies specific for bone morphogenetic protein 2 (BMP-2). In other embodiments, the assays are used to detect non-antibody inhibitors of BMP-2.

In one embodiment, the assay for the detection of neutralizing antibodies to a BMP comprises:
(a) incubating an antibody to be evaluated with a BMP; and
(b) incubating the product of step (a) with BMP-responsive cells containing a BMP-responsive reporter construct.
   The antibody to be evaluated is neutralizing if the level of expression of the reporter gene in the cells of step (b) is less than the level of expression of the reporter gene in the same cells incubated with BMP in the absence of the antibody.

In another embodiment, the assay for the detection of neutralizing antibodies to a BMP comprises the step of incubating an antibody, a BMP, and BMP-responsive cells containing a BMP-responsive reporter construct, wherein the antibody is neutralizing if the level of expression of the reporter gene is less than expression of the reporter gene in the same cells incubated with BMP alone (i.e, without antibody).

In some embodiments, the BMP-responsive cells containing a BMP-responsive reporter construct are prepared by inserting a BMP-responsive reporter construct into a cell line that expresses BMP receptors. In some embodiments, the BMP-reporter construct comprises a luciferase reporter gene driven by the Msx-2 promoter. In another embodiment, the BMP-reporter construct comprises a luciferase reporter gene driven by the ID-1 promoter.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed. The accompanying drawings, which are incorporated in and constitute part of this specification, and together with the description, serve to explain the principles of the invention.

### DETAILED DESCRIPTION OF THE INVENTION

A. Definitions

In order that the present invention may be more readily understood, certain terms are first defined. Additional definitions are set forth throughout the detailed description.

The term “antibody” refers to any immunoglobulin or fragment thereof, and encompasses any polypeptide comprising an antigen-binding site with at least one complimentarity determining region (CDR). The term includes, but is not limited to, polyclonal, monoclonal, monospecific, polyspecific, non-specific, humanized, human, single-chain, chimeric, synthetic, recombinant, hybrid, mutated, grafted, and in vitro generated antibodies. The term “antibody” also includes antibody fragments such as Fab, F(ab′)₂, Fv, scFv, Fd, dAb, and other antibody fragments or other constructs comprising CDRs that retain antigen-binding function. Typically, such fragments would comprise an antigen-binding domain.

The antibody or fragment thereof may be any of the known antibody isotypes and their conformations, for example, IgA, IgG, IgD, IgE, IgM monomers, IgA dimers, IgA trimers, or IgM pentamers.

The term “neutralizing” refers to the ability of a compound or molecule to inhibit (i.e., eliminate or reduce) at least one activity of another compound or molecule. A neutralizing compound may inhibit one or more activities of a molecule without inhibiting other activities of the molecule.

The term “neutralizing antibody” refers to any antibody or fragment thereof capable of binding to and interfering with at least one biological activity of the molecule for which the antibody is specific.

The terms “bone morphogenetic protein” and “BMP” refer to members of the bone morphogenetic protein family, including BMP proteins BMP-2, BMP-3, BMP-4, BMP-5, BMP-6 and BMP-7, disclosed, for example, in U.S. Pat. Nos. 5,108,922, 5,013,649, 5,116,738, 5,106,748, 5,187,076, and 5,141,905, BMP-8, disclosed in PCT WO 91/18098, BMP-9, disclosed in PCT WO 93/00432, BMP-10, disclosed in PCT WO 94/26893, BMP-11, disclosed in PCT WO 94/26892, BMP-12 and BMP-13, disclosed in PCT WO 95/16035, BMP-15, disclosed in U.S. Pat. No. 5,635,372, and BMP-16, disclosed in U.S. Pat. No. 6,331,612, any of the growth and differentiation factors (GDFs) including those described in PCT WO 94/15965, WO 94/15949, WO 95/01801, WO 95/01802, WO 94/21681, WO 94/15966, WO 95/10539, WO 96/01845, WO 96/02559 and others, and MP52, disclosed in PCT WO 93/16099. The disclosures of all of the above patents, and published international applications are incorporated by reference herein.

The term “BMP-2” refers to bone morphogenetic protein 2, a member of the transforming growth factorβ family of growth factors. The term refers to the full-length unprocessed precursor form of BMP-2 as well as the mature and propeptide forms resulting from post-translational cleavage. The term also refers to any fragments and variants of BMP-2 that maintain at least some biological activities associated with mature BMP-2, as discussed herein, including sequences that have been modified. The nucleotide and amino acid sequences of mature human BMP-2 are disclosed in U.S. Pat. No. 5,013,649. The present invention relates to BMP-2 from all vertebrate species, including, but not limited to, human, bovine, chicken, mouse, rat, porcine, ovine, turkey, baboon, and fish. The term BMP-2 includes rhBMP-2, as well as other forms of BMP-2.

The term “rhBMP-2” refers to human BMP-2 produced using recombinant DNA technology.

The term “biological response” refers to any cellular activity induced by a compound such as a BMP. The term “biological response” encompasses the binding of the compound to a cell either directly or via a cellular receptor. The term “biological response” also encompasses internal and external cellular responses, including intracellular signaling events, activation of gene transcription and chemotaxis. The biological response induced by a compound may be a naturally occurring cellular response to the compound or one resulting from materials exogenously added to the cell.

The term “responsive” describes a cell or population of cells capable of mounting a biological response when exposed to a particular compound, such as BMP.

The term “BMP-responsive cell line” refers to a population of cells that expresses BMP receptors and allows the transduction of BMP-induced protein signaling, resulting in the activation of gene expression regulatory elements in response to the interaction between BMP and the cell receptors.

The term “specific binding” refers to two molecules forming a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the affinity constant Kₐ is higher than 10⁶ M⁻¹. If necessary, nonspecific binding can be reduced without substantially affecting specific binding by varying the binding conditions. The appropriate binding conditions, such as concentration of antibodies, ionic strength of the solution, temperature, time allowed for binding, concentration of a blocking agent (e.g., serum albumin, milk casein), etc., may be optimized by a skilled artisan using routine techniques. In the context of BMP-2, however, an antibody or protein specific for BMP-2 may also bind to other BMPs, such as, for example, BMP-4.

B. Assays

One aspect of the present invention involves assays in which a biological response to a BMP is antagonized if a tested sample contains neutralizing antibodies specific for the BMP. The sample containing the potentially neutralizing antibodies is combined with the BMP to allow any potential neutralizing antibodies to bind to the BMP prior to being added to the cell. This combination is then further incubated with a cell capable of biologically responding to the BMP. The biological response induced by the sample/BMP combination is measured and compared with the response induced by the BMP alone. A sample response lower than the response induced by the BMP alone indicates the presence of neutralizing antibodies in the sample.

In one embodiment, the sample and BMP are incubated prior to being added to the cell to allow any potentially neutralizing antibodies to bind to the BMP prior to being added to the cell. In another embodiment, the sample, BMP, and cells are combined at the same time. In yet another embodiment, the BMP and the cells are combined initially, and then the sample is added.

Responses induced by positive and negative control samples are determined to ensure that the assay is functioning properly. Negative controls are typically serum samples from a subject that has not been exposed to the BMP. In some instances, the negative control samples may be pooled serum samples from untreated subjects. Negative control samples measure the biological response induced by the BMP alone. In one embodiment, a reading of biological activity is made from the BMP and cells prior to addition of the sample, serving as a integrated negative control.

Additional controls may include standard solutions containing a range of BMP concentrations. These standards are individually incubated with the cells in the absence of a test sample, thereby generating a profile of biological responses induced at varying BMP concentrations.

Comparison of the response induced by the test sample with those induced by the standards allows for quantitation of the neutralizing activity contained in the test sample. For example, a test sample that induced a response equal to that of a standard containing 50% of the BMP concentration used in the test sample has neutralized 50% of the BMP activity. Units of neutralizing activity may be determined for each test sample using these comparisons.

Any detectable biological response induced by a BMP may be used as an assay readout. BMPs may induce event markers that are early, such as receptor phosphorylation, and late, such as upregulation of cell surface proteins.

1. Bmps

BMPs suitable for the invention include any capable of inducing a biological response when administered to a subject, population of cells, or individual cell. Suitable BMPs are also capable of inducing a humoral immune response when administered to a subject, leading to the production of antibodies specific for the BMP.

Suitable BMPs may be naturally occurring or manufactured using techniques such as recombinant DNA technology.

The assays of the present invention may also be applied to components added to BMPs prior to administration. Examples include excipients, dosage vehicles, and matrices used for drug delivery.

The amount of BMP used in the assay is an amount sufficient to induce the biological response in the cell. However, detection of neutralizing antibodies is most sensitive when the amount of BMP used is not in excess of the amount sufficient to induce the maximum biological response in the cell. BMPs in excess of this amount may bind to neutralizing antibodies within a sample without a detectable change in the assay readout.

The amount of BMP will vary with the parameters of the assay, such as the amount of cells used, the size of the assay vessel and the sensitivity of the cells to the BMP. The amount of BMP can be titrated to determine the optimal amount sufficient to induce the biological response in the cell without saturating the assay with excess BMP.

In alternative embodiments, the concentration of BMP used in the assay may be the following: at least 1 pg/mL, at least 100 pg/mL, at least 500 pg/mL, at least 1 ng/mL, at least 100 ng/mL, at least 200 ng/mL, at least 500 ng/mL, at least 1 μg/mL, at least 100 μg/mL, at least 500 μg/mL, or at least 1 mg/mL.

In some embodiments of the invention, the BMP may be BMP-1 to BMP-18 or MP-52. In other embodiments, the BMP is BMP-2. In additional embodiments, the BMP is rhBMP-2.

For purposes of illustration only, the invention was applied to neutralizing antibodies specific for recombinant human bone morphogenetic protein 2 (rhBMP-2).

BMP-2 is a member of the transforming growth factor-β(TGF-β) family of growth factors. BMP-2 is capable of inducing new bone formation, particularly when surgically implanted on a matrix. rhBMP-2, recombinantly produced human BMP-2, is of clinical interest for, among other things, its ability to facilitate the repair of bone fractures.

Structurally, BMP-2 is a homodimer with two distinct sites that interact with receptors. BMP-2 stimulation of responsive cells occurs through two cellular transmembrane serine/threonine kinase receptors, termed type I and type II receptors. One site on BMP-2 interacts with the high affinity binding type I receptor and spans the interface of the BMP-2 dimer. The second site, smaller and located on each BMP-2 monomer, interacts with the low affinity binding type II receptor.

To date, there are three known type I receptors (designated activin receptor-like kinases Alk-3, Alk-6 and ActR-1) and two known type II receptors (designated BMPR-II and ActR-IIA). The type II receptors are constitutively phosphorylated, while type I receptors are phosphorylated following BMP-2 stimulation of cells. The BMP-2 signal cascade is initiated when BMP-2 binds to a type I receptor that then recruits a type II receptor. The type II receptor activates the type I receptor by transphosphorylation. The activated type I receptor can in turn phosphorylate members of the SMAD protein family. SMAD proteins translocate to the cell nucleus and activate more specific genes in the BMP-2 regulated pathway.

The transcription factor Msx-2 is a member of the homeobox gene family and plays an important role in bone formation. See, e.g., Hruska et al., Circ. Res., 97:105-114 (2005). In cells stimulated by BMP-2, Msx-2 is rapidly upregulated and thus serves as an early marker for BMP-2 signal transduction. The promoter sequence for the Msx-2 gene has been characterized. See Liu et al., Proc. Natl. Acad. Sci., 92:6137-41 (1995).

The transcription factor ID-1 is a dominant negative inhibitor of basic helix-loop-helix proteins and is a direct target of BMP. BMP strongly activates the ID-1 promoter and ectopic expression of ID proteins can mimic BMP-induced responses. The ID-1 promoter contains two distinct BMP-specific responsive regions. Korchynskyi et al., J Biol Chem, 277(7)4883-91(2002). Accordingly, the ID-1 promoter may be used as an alternative to the Msx-2 promoter in the assays and cells of the invention.

In contrast to Msx-2 and ID-1, BMP-2 stimulation of responsive cells induces the production of alkaline phosphatase after a period of hours. Alkaline phosphatase thus serves as a late marker for BMP-2 signal transduction. See, e.g., Thies et al., Endocrinology, 130:1318-24 (1992).

In selecting a biological system to detect neutralizing antibodies to rhBMP-2, consideration was given to whether the marker to be followed was related to an early or late event in the signal process pathway. This rationale was based on the advantages an early event offers in terms of specificity and speed of read out. A late event could potentially be triggered through other receptor-mediated pathways, which would contribute to the background signal. This signal could be reduced by inhibitors other than neutralizing antibodies to BMP-2, resulting in false negative results. Once a marker, a cell line and a BMP-2-neutralizing positive control protein were identified, additional experiments were conducted to further characterize the system.

BMPs exert a physiological effect by inducing a biological response in a responsive cell. For example, a BMP may bind to a cell receptor and initiate a cascade of signaling events leading to the upregulation of gene transcription. For example, BMP-2 binding by responsive cells leads to events such as receptor phosphorylation, increased transcription of the Msx-2 gene and upregulation of alkaline phosphatase activity. These biological responses may serve as readouts for the assays of the invention.

In one embodiment of the invention, the assay measures an endogenous biological response in cells responsive to rhBMP-2. In another embodiment, the assay measures the activation of a reporter construct inserted into cells responsive to rhBMP-2.

Many BMPs induce a biological response by binding to a target molecule in vivo, such as a receptor present on the surface of the cell. The assays of the present invention may involve the disruption of this binding interaction. These assays measure the ability of a test sample to inhibit (i.e., eliminate or reduce) the binding of the BMP to its physiological ligand.

In one embodiment of the invention, the ligand may be present on a cell or population of cells. In another embodiment, the ligand may be used in soluble form in a cell-free assay. In another embodiment, the ligand may be bound to a solid support.

In one aspect, the test sample is incubated with a BMP labeled with a detectable marker. The mixture is then incubated with the ligand so that the BMP and ligand may bind. The amount of BMP specifically bound to the ligand may then be determined by detection of the marker.

Examples of detectable markers suitable for use in the invention include biotin, fluorescent labels, enzyme labels, radioisotopes, chemiluminescent labels, haptens, dyes and others. The markers may be detected by any means known in the art, such as spectrophotometry, radiography or flow cytometry.

2. Cells and Cell Culture Conditions

In certain embodiments of the invention, the assays are cell-based assays. Cells used in the assays of the invention are capable of responding to the BMP being administered. The assays of the invention may use one cell or a population of cells.

In one embodiment, the assay is conducted on a single cell. In other embodiments, the assay is conducted on more than one cell.

Cells are grown at any density appropriate for normal cell growth when used in the assays of the invention. The number of cells used to achieve an appropriate density is determined in part by the size and surface area of the tissue culture dish or plate used in the assay.

Cells may be used in the assay at any density. In alternative embodiments, the cells are used in the assay at the following cell densities: at least 10% confluent, at least 25% confluent, at least 50% confluent, at least 80% confluent, at least 90% confluent, or at least 99% confluent.

In other alternative embodiments, the cells are used in the assay at the following concentrations: at least 1×10³/ mL, at least 5×10³/mL, at least 1 ×10⁴/mL, at least 5×10⁴/ mL, at least 1×10⁵/ mL, at least 5×10⁵/mL,at least 1×10⁶/mL, at least 5×10⁶/ mL or at least 1×10⁷/mL.

In certain embodiments, the cells are naturally capable of responding to the BMP. In other embodiments, the cells have been manipulated to alter the response induced by the BMP. In additional embodiments, a reporter construct has been introduced into the cells, for example, by transfection.

In one aspect of the invention, the cells are cell lines; in other aspect, the cells are cells isolated from a subject and cultured ex vivo.

The cells of the present invention may be mammalian or non-mammalian. The cells may be of any species, including, but not limited to, human, monkey, mouse, rat, hamster and other vertebrate species. The cells may also be from invertebrate species such as, for example, insect cells. The cells may also be prokaryotic cells such as, for example, bacterial cells.

Cells may be cultured according to any of techniques known in the art. Cells may be grown in any culture flask, plate or dish suitable forcell culture. Media, supplements and culture conditions appropriate for cell culture are well known to those of skill in the art.

In one embodiment of the invention, the cells are capable of binding and responding to BMP-2. Examples of BMP-2 responsive cells include the murine stromal cell line W-20, the murine limb bud cell lines MLB13myc-c14 (C14) and C36, and the murine cell line C2C12. These cell lines all respond to rhBMP-2 stimulation by the activation of signal transduction pathways leading to receptor phosphorylation, increased transcription of the Msx-2 gene and upregulation of alkaline phosphatase.

The C36 cell line was created by transfecting the MLB13myc-c14 cell line with a construct containing the Msx-2 promoter sequence directly upstream from the luciferase gene sequence. Due to the presence of the Msx-2 promoter, luciferase will be produced in C36 cells whenever Msx-2 gene transcription is initiated. Therefore, upon binding rhBMP-2, C36 cells will rapidly express luciferase.

In one embodiment of the invention, the cells are C36 cells. In another embodiment, the cells are a cell line derived from C36 cells.

In one embodiment of the invention, the cells are W-20 cells. In another embodiment, the cells are a cell line derived from W-20 cells.

3. Samples

Test samples taken from a subject may be of any bodily fluid capable of containing neutralizing antibodies or proteins. Examples include, but are not limited to, blood, serum, lymph, plasma, synovial fluid, cerebrospinal fluid, lachrymal fluid, biopsy or tissue sample, cell suspension, saliva, oral fluid, mucus, amniotic fluid, colostrums, mammary gland secretions, urine, sweat and tissue culture medium.

Test samples may be assayed at multiple dilutions to obtain an accurate quantitation of neutralizing activity present in the sample. The units of neutralizing activity in each sample may be calculated based on the amount each sample was diluted.

Test samples may also be diluted to avoid interference from non-specific background components of the samples (i.e., matrix interference). For example, proteins found at high concentrations in the serum may, in some circumstances, non-specifically interact with components of the assay and reduce the sensitivity of the assay. Sample dilution may reduce or eliminate non-specific binding and thereby increase the signal-to-noise ratio of the assay.

In one embodiment, samples are assayed undiluted. In other alternative embodiments, samples are assays at dilution factors such as, for example, 1:1, 1:2, 1:5, 1:10, 1:15, 1:20, 1:30, 1:50, 1:60, 1:100, 1:500, 1:1000, or 1:5000. In additional embodiments, the samples are assayed at still greater dilution factors.

In one embodiment of the invention, samples are initially assayed at a constant dilution factor. Samples testing positive for neutralizing activity are then assayed at a range of dilution factors to better quantitate the amount of neutralizing activity present in each sample.

In some embodiments of the invention, the assays are conducted on 96-well plates containing the test samples and the control samples on the same assay plate. In other embodiments, the assays are conducting by high-throughput screening.

One aspect the present invention involves assays for detecting neutralizing antibodies in the serum of a subject. Prior to testing, serum samples may be taken from a subject by any means known in the art. Serum samples may also be heat inactivated and delipidated, or otherwise treated, by conventional techniques prior to use in the assays of the invention.

Serum samples may also be prescreened to determine if antibodies specific for the BMP of interest are present prior to determining whether the antibodies are neutralizing. In one embodiment, the serum samples are prescreened with an ELISA assay; in another embodiment, the serum samples are prescreened by western blotting.

In one embodiment, the serum samples are prescreened for antibodies specific for BMP-2; in an additional embodiment the serum samples are prescreened for antibodies specific for rhBMP-2.

The assays of the inventions are capable of detecting neutralizing antibodies specific for the BMP as well as non-antibody molecules capable of inhibiting the activity of the BMP. The assays may be combined with additional tests to confirm the presence of antibodies in general and antibodies specific for the BMP in particular. Tests for the detection of antigen-specific antibodies are well known in the art.

In one embodiment, the assay samples are also tested by ELISA. In another embodiment, the assay samples are also tested by western blotting.

4. Positive Controls

Positive control samples include a component known to be specifically capable of neutralizing the activity of the BMP. Positive controls may include antibodies specific for the BMP, such as monoclonal antibodies (mAbs) or antibody-positive sera from animals treated or immunized with the BMP.

In one embodiment, the positive control is a monoclonal antibody; in another embodiment, a polyclonal antibody; in another embodiment, an antibody fragment.

In alternative embodiments of the invention, positive controls are antibodies specific for the rhBMP-2, such as monoclonal antibodies (mAbs), polyclonal antibodies or antibody-positive sera from animals treated or immunized with rhBMP-2. In one embodiment of the invention, the positive control is the mAb MAB3552 (R&D Systems, Minneapolis, Minn.).

Positive controls may also include non-antibody molecules capable of neutralizing the activity of the BMP. For example, a natural antagonist capable of specifically binding to and inhibiting the biological activity of the BMP may be used.

In one embodiment, the positive control is a biological ligand for the BMP. In another embodiment, the positive control is a receptor capable of binding to the BMP.

Positive controls may also include non-antibody molecules capable of neutralizing the activity of rhBMP-2. For example, a molecule capable of binding to and inhibiting the biological activity of rhBMP-2 may be used.

A number of antagonists of BMP family members have been described. See, e.g., Yanagita, Cytokine Growth Factor Rev., 16:309-17(2005). This reference discloses several molecules that bind to and antagonize BMP-2 activity, and are thus suitable positive controls for one embodiment of the invention. Among these is noggin, a small glycoprotein protein capable of binding BMP-2 with high affinity.

The biological activity and amino acid sequence of noggin is highly conserved among species. The amino acid sequences of several species of noggin have been published. See, e.g., Valenzuela et al., J. Neurosci., 15:6077-84 (1995). Purified murine noggin is available from R&D Systems (Minneapolis, Minn.).

In one embodiment, the positive control is a biological ligand for rhBMP-2. In another embodiment, the positive control is an antagonist of rhBMP-2 binding to its cellular receptor. In another embodiment, the positive control is the protein chordin. In still another embodiment, the positive control is noggin.

Noggin molecules suitable for use in the present invention may be from any species, including mammalian and non-mammalian species, such as, for example, human, mouse, monkey and frog. Homologs of noggin from these and other species are also suitable positive controls.

The amount of positive control used in the assays of the invention is an amount sufficient to inhibit (i.e., eliminate or reduce) the activity of the BMP. The amount of positive control will vary with the parameters of the assay, such as the amount of cells used, the size of the assay vessel and the sensitivity of the cells to the BMP. The amount of positive control can be titrated to determine the optimal amount sufficient to inhibit the activity of the BMP without saturating the assay with excess positive control.

One way to assess the amount of positive control used in the assay is to measure the affinity of the positive control for the BMP. Generally, the greater the affinity of the positive control for the BMP, the less positive control is needed in the assay. In one embodiment of the invention, the positive control and BMP are used in equimolar amounts in the assay.

In alternative embodiments, the concentration of positive control used in the assay may be the following: at least 1 pg/mL, at least 100 pg/mL, at least 500 pg/mL, at least 1 ng/mL, at least 100 ng/mL, at least 500 ng/mL, at least 1 μg/mL, at least 100 μg/mL, at least 500 μg/mL, at least 1 mg/mL, or at least 100 mg/mL.

The positive control molecule may need to be produced in soluble form for use in the assay. For example, the ligand-binding portion of a membrane-bound receptor may be produced as a soluble fusion protein for use as a positive control. The positive control may be used in the assay in soluble form, or may be further attached to a solid support, such as a bead or plate.

5. Assay Readouts

In selecting a biological response for use in the assays of the invention, consideration must be given to whether the marker to be followed is related to an early or late event in the signal process pathway. Early events may offer increased specificity and speed of read out. A late event could potentially be triggered through other receptor-mediated pathways, which would contribute to the background signal. This signal could be reduced by inhibitors other than neutralizing antibodies to the BMP, resulting in false negative results. Once a marker, a cell line and a positive control protein are identified, additional experiments may be conducted to further characterize the system.

Biological event markers may occur naturally in cells upon exposure to a BMP. For example, a cell may produce an enzyme in response to stimulation with a BMP. Alternatively, a reporter system may be introduced into the cell for use as an assay readout. For example, a reporter gene may be linked to the promoter of a gene activated upon exposure of the cell to the BMP and introduced into the cell. Exposure of the cells to the BMP will result in activation of the reporter gene. Multiple copies or the promoter and/or reporter gene may be included on the reporter construct to increase the assay signal.

In one embodiment, the reporter gene is luciferase. In another embodiment, the reporter gene encodes a fluorescent protein. In another embodiment, the reporter gene is green fluorescent protein.

Any gene upregulated in a cell by exposure to a BMP may be used as an assay readout. Reporter gene constructs may include the promoter sequence from any gene upregulated in a cell in response to the BMP.

Examples of potential biological events for use as assay readouts include receptor binding, receptor phosphorylation, markers indicative of the activation of gene transcription, upregulation of cell surface molecules, activation of a reporter gene, consumption of growth factors, changes in growth media components or parameters (e.g., pH, dissolved O₂, CO₂ production), secretion of proteins or enzymes, dephosphorylation events, calcium influx, cellular proliferation, apoptosis, changes in mitochondrial or metabolic markers, and morphological changes.

Detection methods for assay readouts are well known in the art. Examples of detection methods include immunoassays such as ELISA assays, chemiluminescence, western blotting, flow cytometry, spectrophotometry, colorimetry, radiography, microscopy, surface plasmon resonance, PCR, RT-PCR, and gel electrophoresis.

In one embodiment of the invention, the assay detects neutralizing antibodies specific for rhBMP-2. Examples of readouts for this assay include the binding of rhBMP-2 to type I and type II BMP-2 receptors, the phosphorylation of these receptors and the production of alkaline phosphatase.

In one embodiment, a gene upregulated by exposure of a cell to rhBMP-2 is monitored by measuring mRNA levels by RT-PCR. Increased gene is expression is normalized to the expression level of a housekeeping gene such as GAPDH, β-actin or 18S rRNA. Examples of genes upregulated by a cell exposed to rhBMP-2 include, for example, fibromodulin, Smad 6, Msx-2, Hey 1, SFRP-2, and SFRP-2.

In a further embodiment, the assay readout is the luciferase activity induced in C36 cells upon exposure to rhBMP-2. In one embodiment, luciferase activity is detected by chemiluminescence.

6. Data Analysis

Data analysis can be performed using a variety of means. For example, the neutralizing activity of samples and positive controls may be assessed using units of neutralizing activity (UNA) values. The UNA value is related to the percentage of biological activity that is neutralized by a sample. When the assay readout is chemiluminescence, by definition, a sample that reduces the activity of 200 ng/mL of the BMP by exactly 50% (i.e., the CPS generated by a sample incubated with 200 ng/mL rhBMP-2 equals the CPS of 100 ng/mL standard) has 50 units of neutralizing activity. If a sample reduces the activity by greater than 50%, the reported neutralizing activity for that sample is calculated by multiplying 50 UNA by a correction factor based on the dilution of that sample. Any sample that reduces the activity of 200 ng/mL of the BMP by less than 50% (i.e., CPS generated by a sample incubated with 200 ng/mL of the BMP is greater than the CPS of 100 ng/mL standard) is considered negative.

If the sample average CPS value is less than or equal to the CPS value for the 100 ng/mL of the BMP standard, the sample is called positive. To determine the UNA value for the positive control, the sample may be analyzed at multiple dilutions. The sample dilution at which the corresponding CPS value is equal to that for the 100 ng/mL of the BMP standard is interpolated by the following equation:

$$Dilution = DilnCPS 1 - [ \frac{( CPS 1 - BP 100 )}{CPS 1 - CPS 2} \times ( DilnCPS 1 - DilnCPS 2 ) ]$$

Where BP100(BMP)=raw signal value (CPS) generated by the 100 ng/mL of the BMP standard, CPS1=sample CPS value above the 100 ng/mL of the BMP standard CPS in the dilution series, CPS2=sample CPS value below the 100 ng/mL of the BMP standard CPS in the dilution series, DilnCSP1=sample reciprocal dilution at CPS1, and DilnCSP2=sample reciprocal dilution at CPS2.

Reportable sample UNA value is calculated by multiplying 50 UNA by a correction factor that is based on the sample dilution that would result in 50% reduction of activity according to the following equation:
Reported UNA=50 UNA×LOG(Diln100)/LOG(60)
Where Diln 100=the sample dilution where the signal equals the signal of the 100 ng/mL of the BMP standard and 60 is the sample dilution.

Analogous calculations may be performed for different assay readouts, substituting the different biological responses and readouts for luminescence and CPS.

### EXAMPLES

The following examples are offered for illustrative purposes only.

### Example 1

Construction of the Msx-2-Luciferase Reporter

A 2 kb BamHI fragment of the murine Msx-2 promoter (described in Liu et al., Proc. Natl. Acad. Sci., 92:6137-41 (1995)) was cloned into the pGL2-basic vector (Promega Corp., Madison, Wis.) in a position upstream of the luciferase gene. This construct drives BMP-2 induced expression of luciferase in cells responsive to BMP-2. See Daluiski et al., Nature Genet., 27:84-88 (2001).

### Example 2

C36 Cell Line Construction

The murine limb bud cell line MLB13myc-c14 was chosen for the assay due to its responsiveness to rhBMP-2. See Rosen et al., J. Bone Miner. Res., 9:1759-68 (1994). MLB13myc-c14 cells were co-transfected with two vectors: the Msx-2-luciferase construct described in Example 1 and a plasmid containing the hygromycin resistance gene driven by the thymidine kinase promoter. Transfections were conducted using Lipofectamine 200 reagent (Invitrogen, Carlsbad, Calif.). Cells were then grown in media containing 40 μg/mL hygromycin to select for those stably expressing the co-transfected constructs. The resulting cell line was designated C36.

Assessment of the types of receptors on the surface of C36 cells was done by western blot using commercially available anti-Alk3, anti-Alk-6, anti-BMPRII, anti-ActRIIb (R&D Systems, Minneapolis, Minn.), but no specific binding was observed on cell lysates. From the literature it is well known that at least one type I receptor and one type II receptor are present on the cell surface if on BMP-2 stimulation the cell is able to produce the enzyme alkaline phosphatase. Since all the cells evaluated could produce alkaline phosphatase, the C36 cells express at least one type I and one type II surface receptor.

### Example 3

Positive Control Selection

Development and optimization of a bioassay for the detection of neutralizing antibodies to rhBMP-2 required a suitable positive control with neutralizing activity. Potential sources for positive controls included antibody-positive sera from animals treated or immunized with rhBMP-2 or monoclonal antibodies (mAbs) specific for rhBMP-2. Sera from rhBMP-2-treated monkeys and an immunized rabbit as well as purified monoclonal antibodies were assessed for neutralizing antibody activity.

The murine stromal cell line W-20 upregulates alkaline phosphatase activity in response to treatment with rhBMP-2. See Thies et al, Endocrinology, 130:1318-24 (1992). The ability of the antibodies to inhibit the activity of BMP-2 was tested by coincubating W-20 cells with BMP-2 and each antibody and then examining the alkaline phosphatase activity of the cells. Human noggin, a natural soluble ligand for BMP-2, was also assayed for the ability to inhibit BMP-2 activity. The antibodies and noggin were also evaluated for their ability to inhibit the activity of rhBMP-2 in C36 cells.

The monoclonal antibody MAB3552 (R&D Systems, Minneapolis, Minn.) was found to have rhBMP-2 neutralizing activity and was identified as a secondary positive control. Noggin was found to be the best antagonist of BMP-2 activity, and was thus chosen as the primary positive control.

### Example 4

Preparation of Human Serum Samples

Human serum samples were isolated from subjects and heat inactivated by incubation at 56° C. for 30 minutes. The samples were then delipidated by treatment with 1:10 weight/volume of silica (Sigma, St. Louis, Mo.) for 30 minutes at room temperature with rotation. The silica was removed by filtration using a sterile spin cup 0.2 micron filter unit placed inside of a sterile 0.5 mL centrifuge tube. The tubes were centrifuged for 1 to 2 minutes at approximately 14,000 rpm in an Eppendorf centrifuge. All serum samples were handled under sterile conditions in sterile hoods.

### Example 5

Preparation of Noggin Positive Controls

Human noggin was used as a positive control. Human noggin was cloned and produced by conventional recombinant techniques. The nucleotide and amino acid sequence of human noggin have been published. See, e.g., Valenzuela et al., J. Neurosci., 15:6077-84 (1995).

Noggin positive control stock solutions were prepared at various concentrations in treated normal human serum (T-NHS; heat inactivated and delipidated pooled human serum).

### Example 6

Preparation of rhBMP-2 Standards

Intermediate rhBMP-2 standards were used to prepare final rhBMP-2 solutions for incubation with the cells in 96 well plates. Intermediate solutions were prepared in treated normal human serum (T-NHS; heat inactivated and delipidated pooled human serum) as outlined in Table I. Specifically, the amount of rhBMP-2 stock solution indicated in column D (concentration provided in column C) was diluted with the amount of T-NHS indicated in column E to produce the intermediate solution indicated in column B. Intermediate solutions were then diluted by mixing 67 μL of each solution with 933 μL of the complete medium without phenol red, resulting in the final assay concentration indicated in column A.

TABLE 1
Preparation of rhBMP-2 Standards

| A | | | | |
|---|---|---|---|---|
| Final rhBMP-2 | B | C | D | |
| concentration | Intermediate | rhBMP-2 stock | Volume of | E |
| co-incubated | solution of | solution used | rhBMP-2 | Volume of |
| with the cells, | rhBMP-2 | in preparation | stock solution | T-NHS added |
| (ng/mL) | (μg/mL) | (μg/mL) | added (μL) | (μL) |
| 400 | 12.0 | 100 | 30.0 | 220 |
| 300 | 9.0 | 100 | 30.0 | 303 |
| 200 | 6.0 | 100 | 30.0 | 470 |
| 180 | 5.4 | 100 | 13.5 | 237 |
| 170 | 5.1 | 100 | 12.8 | 237 |
| 160 | 4.8 | 100 | 24.0 | 476 |
| 120 | 3.6 | 100 | 18.0 | 482 |
| 100 | 3.0 | 6.0 | 200 | 200 |
| 80 | 2.4 | 4.8 | 200 | 200 |
| 60 | 1.8 | 3.6 | 200 | 200 |
| 50 | 1.5 | 3.0 | 200 | 200 |
| 40 | 1.2 | 2.4 | 200 | 200 |
| 20 | 0.6 | 1.2 | 200 | 200 |
| 0 | 0 | Not Applicable | 0.0 | 100 |

Concentrations of the rhBMP-2 standards that were chosen for the final assay format were 200, 100 and 0 ng/mL.

### Example 7

rhBMP-2 Neutralizing Antibody Assay

All cells were cultured under sterile conditions with supplemented Dulbecco's Modified Eagle Media (DMEM). Immediately prior to plate luminescence readings, cells were cultured in DMEM without phenol red to prevent signal interference.

C36 cells were thawed and grown in flasks for 3 days until approximately 80% confluent. Cells were then removed from the flask using trypsin, seeded into a 96-well flat-bottom plate at approximately 8×10⁴ cells/mL, and grown overnight at 7.5% CO₂ and 37° C. The cells were approximately 80% confluent following overnight incubation. Human serum samples, noggin positive control samples or rhBMP-2 standards, prepared as described above, were spiked with rhBMP-2 and the mixture was incubated for one hour. Unless indicated, the samples were spiked with 200 ng/mL rhBMP-2.

The spiked samples were added to the C36 cells and incubated overnight. The media was then removed by aspiration and the plates were developed under subdued lighting. BriteLite substrate (PerkinElmer, Boston. Mass.) containing a detergent lysis buffer was added to each well. The plate was then shaken for one minute and the luciferase luminescence signal was read on a Victor II luminescence counter. A decrease in signal with the noggin positive control and rhBMP-2 relative to rhBMP-2 alone was detected in this manner and reported as percent inhibition of the pre-defined rhBMP-2 concentration. The pre-defined concentration of rhBMP-2 used was 80% of the maximum signal plateau.

To control plate performance, each sample plate included a set of rhBMP-2 standards and a set of noggin positive control solutions. Plates were rejected if raw signal for the 100 ng/mL rhBMP-2 standard was higher than 90% of the signal generated by the 200 ng/mL rhBMP-2 standard.

### Example 8

Data Analysis

All samples were initially diluted 1:60 and spiked with 200 ng/mL of rhBMP-2. Luciferase signal values (counts per second, CPS) produced in the assay for each sample was analyzed relative to the performance of the rhBMP-2 standards.

The neutralizing activity of samples and positive-controls was assessed using units of neutralizing activity (UNA) values. The UNA value is related to the percentage of rhBMP-2 activity that is neutralized by a sample. By definition, a sample that reduces the activity of 200 ng/mL of rhBMP-2 by exactly 50% (i.e., the CPS generated by a sample incubated with 200 ng/mL rhBMP-2 equals the CPS of 100 ng/mL standard) has 50 units of neutralizing activity. If a sample reduces the activity by greater than 50%, the reported neutralizing activity for that sample is calculated by multiplying 50 UNA by a correction factor based on the dilution of that sample. Any sample that reduces the activity of 200 ng/mL rhBMP-2 by less than 50% (i.e., CPS generated by a sample incubated with 200 ng/mL rhBMP-2 is greater than the CPS of 100 ng/mL standard) was considered negative.

If the sample average CPS value was less than or equal to the CPS value for the 100 ng/mL rhBMP-2 standard, the sample was called positive. To determine the UNA value for the positive control, the sample was analyzed at multiple dilutions. The sample dilution at which the corresponding CPS value was equal to that for the 100 ng/mL rhBMP-2 standard was interpolated by the following equation:

$$Dilution = DilnCPS 1 - [ \frac{( CPS 1 - rhBMP 100 )}{CPS 1 - CPS 2} \times ( DilnCPS 1 - DilnCPS 2 ) ]$$

Where rhBMP100=raw signal value (CPS) generated by the 100 ng/mL rhBMP-2 standard, CPS1=sample CPS value above the 100 ng/mL rhBMP-2 standard CPS in the dilution series, CPS2=sample CPS value below the 100 ng/mL rhBMP-2 standard CPS in the dilution series, DilnCSP1=sample reciprocal dilution at CPS1, and DilnCSP2=sample reciprocal dilution at CPS2.

Reportable sample UNA value was calculated by multiplying 50 UNA by a correction factor that was based on the sample dilution that would result in 50% reduction of activity according to the following equation:
Reported UNA=50 UNA×LOG(Diln100)/LOG(60)
Where Diln 100=the sample dilution where the signal equals the signal of the 100 ng/ml rhBMP-2 standard and 60 is the sample dilution.

### Example 9

Calibration Curve Range and Selection of rhBMP-2 Calibration Standards

Initially, the rhBMP-2 response curve was evaluated to determine the optimum rhBMP-2 concentration to be spiked into samples and controls for analyzing neutralizing activity. For this experiment, the final concentration of rhBMP-2 incubated with the cells was varied between 400 ng/mL and 0 ng/mL. Table 2 shows the CPS signals for each rhBMP-2 concentration tested in the C36 assay, as outlined above.

TABLE 2
Average rhBMP-2 Dose Response

| rhBMP-2 | Mean Raw Signal | Standard | |
|---|---|---|---|
| (ng/mL) | (CPS) | Deviation | % CV |
| 400 | 45850 | 6143 | 13.4 |
| 300 | 45844 | 6844 | 14.9 |
| 200 | 43424 | 6034 | 13.9 |
| 180 | 42936 | 6978 | 16.3 |
| 170 | 41525 | 5808 | 14.0 |
| 160 | 42032 | 6530 | 15.5 |
| 120 | 39590 | 6069 | 15.3 |
| 100 | 39933 | 6616 | 16.6 |
| 80 | 36318 | 4486 | 12.4 |
| 60 | 33439 | 6219 | 18.6 |
| 50 | 33890 | 4948 | 14.6 |
| 40 | 33568 | 4584 | 13.7 |
| 20 | 30426 | 4088 | 13.4 |
| 0 | 24833 | 3700 | 14.9 |

N = 39

Since CPS signal starts to plateau at approximately 200 ng/mL of BMP-2, this standard concentration was selected as the rhBMP-2 concentration to be spiked into all samples and positive control solutions. All the standards were evaluated and the following acceptance criteria were selected for the validation plates: a) the raw signal (CPS)for the 100 ng/mL rhBMP-2 standard should be less than or equal to 90% of the CPS for the 200 ng/mL standard, and b) the raw signal (CPS) for the 0 ng/mL rhBMP-2 Standard should be less than or equal to 90% of the CPS for the 100 ng/mL standard.

The performance of the 200, 100 and 0 ng/mL rhBMP-2 standards in the C36 assay were evaluated for variability when tested on different days. As shown in Table 3, the total inter-day variability (%CV) of the calibration standards was less than 20%.

TABLE 3
Inter-day Variability of the rhBMP-2 Standards

| | 200 ng/mL | 100 ng/mL | 0 ng/mL |
|---|---|---|---|
| Sample | rhBMP-2 | rhBMP-2 | rhBMP-2 |
| 1 | 53657 | 46276 | 23566 |
| 2 | 53391 | 43881 | 22655 |
| 3 | 44540 | 38700 | 21030 |
| 4 | 54076 | 39987 | 22273 |
| 5 | 42952 | 37813 | 18636 |
| 6 | 38415 | 28787 | 18215 |
| 7 | 41184 | 35026 | 23389 |
| 8 | 41043 | 35493 | 24737 |
| 9 | 41467 | 34823 | 25342 |
| 10 | 41536 | 35177 | 25111 |
| 11 | 34633 | 28011 | 18211 |
| 12 | 40881 | 33397 | 22762 |
| 13 | 49195 | 40426 | 25843 |
| 14 | 41692 | 32187 | 21312 |
| 15 | 58918 | 44155 | 26005 |
| 16 | 59606 | 37971 | 24159 |
| Mean | 46074 | 37007 | 22703 |
| Std. Deviation | 7624 | 5218 | 2618 |
| % CV | 16.5 | 14.1 | 11.5 |

### Example 10

Inhibition by Noggin and MAB3552

The neutralizing activity of MAB3552 and noggin were compared with a Monoclonal antibody that does not interact with BMP-2 as a negative control (Negative Control Ab). Table 4 shows the percentage inhibition of rhBMP-2 activity at varying molar ratios of inhibitor to rhBMP-2 used in the C36 assay described above. The results indicate that both noggin and MAB3552 possess rhBMP-2 neutralizing activity and could be used as positive controls for the assay.

TABLE 4
Percentage Inhibition by Noggin at Different Molar Ratios

| | | | Negative |
|---|---|---|---|
| Molar Ratio of | Noggin + | MAB3552 + | Control Ab + |
| Inhibitor to rhBMP-2 | rhBMP2 | rhBMP-2 | rhBMP-2 |
| 1 | 101% | 26% | 10% |
| 10 | 105% | 44% | 0% |
| 100 | 101% | 86% | 15% |

Noggin samples were spiked with 132 ng/mL rhBMP-2 and incubated for one hour prior to addition to the C36 cells. Following overnight culture, the luciferase activity of the C36 cells was determined as explained above. Table 5 indicates the percent inhibition of rhBMP-2 activity at final noggin concentrations of 100, 25, 6.25 and 1.5 ng/mL. Results showed that the noggin positive control inhibited rhBMP-2 between 56-100%.

TABLE 5
Percentage Inhibition by Noggin Samples

| | 100 ng/mL | 25 ng/mL | 6.25 ng/mL | 1.5 ng/mL |
|---|---|---|---|---|
| Sample | Noggin | Noggin | Noggin | Noggin |
| 1 | 100% | 86% | 51% | 58% |
| 2 | 100% | 80% | 35% | 50% |
| 3 | 100% | 87% | 66% | 63% |
| 4 | 100% | 87% | 59% | 65% |
| 5 | 100% | 82% | 54% | 54% |
| 6 | 100% | 87% | 51% | 58% |
| 7 | 98% | 93% | 77% | 74% |
| 8 | 100% | 79% | 53% | 53% |
| Mean | 100% | 85% | 56% | 59% |
| % CV | 1 | 5 | 22 | 13 |

### Example 11

Effect of Sample Pretreatment

Serum components such as complement or lipids have the potential to interfere in a cell-based assay. Typical methods applied to minimize interference by these components include heat inactivation and delipidation treatment. Duplicate normal pooled and individual human samples were evaluated for interferences in the C36 cell assay without spiked rhBMP-2. It was noted that the variability between duplicate samples (%CV) was above 25%, as shown in Table 6.

TABLE 6
Variability of Duplicate Human Serum Samples in the
C36 Cell-Based Bioassay

| | | Visible | | % CV |
|---|---|---|---|---|
| Serum | Serum | Condition | | Between |
| Sample ID | Source | of Serum | Mean CPS | Duplicates |
| NHS 148 | Individual | Clear | 2486 | 26 |
| NHS 01E0512 | Pool | Cloudy | 5427 | 27 |
| NHS 146 | Individual | Milky | 5023 | 31 |
| NHS 147 | Individual | Slightly Cloudy | 3750 | 27 |

To evaluate whether heating and lipid removal treatment of serum samples would reduce the variability of the duplicate values, fifteen individual human serum samples were pretreated by heat inactivation and delipidation as described in Example 4. Duplicate samples were evaluated for interferences in the C36 cell assay with or without spiked rhBMP-2. The results are shown in Table 7.

TABLE 7
Effect of Sample Pretreatment on the Variability
of Duplicate Human Serum Samples

| | | % CV | Mean CPS | % CV |
|---|---|---|---|---|
| | Mean CPS (No | Between | (250 ng/mL | Between |
| Sample | rhBMP-2) | Duplicates | rhBMP-2) | Duplicates |
| 1 | 7728 | 2 | 14517 | 14 |
| 2 | 7803 | 2 | 15615 | 4 |
| 3 | 7311 | 18 | 15505 | 7 |
| 4 | 7337 | 1 | 15729 | 2 |
| 5 | 7354 | 2 | 14157 | 8 |
| 6 | 6430 | 20 | 14065 | 18 |
| 7 | 7051 | 12 | 14781 | 15 |
| 8 | 7272 | 22 | 15358 | 20 |
| 9 | 6496 | 23 | 13984 | 16 |
| 10 | 6233 | 29 | 11762 | 8 |
| 11 | 7053 | 11 | 13056 | 0 |
| 12 | 6796 | 15 | 13742 | 4 |
| 13 | 7860 | 16 | 14433 | 14 |
| 14 | 6034 | 12 | 12326 | 1 |
| 15 | 6177 | 12 | 12266 | 9 |
| | Average % CV | 13 | Average % CV | 9 |

The results indicate that following sample pretreatment, the variability of 97% of the samples was below 25%. The pretreatment had no effect on the neutralizing activity of monoclonal antibody MAB3552 when added to the sample (not shown). In addition, cellulose acetate or low binding PVDF filters were both used successfully to remove silica and had no effect on the neutralizing activity of MAB3552.

### Example 12

Analysis of Positive Control and Blank Solution

Intra- and inter-day variability of the positive control noggin solutions in the C36 assay was evaluated. A 72 μg/mL noggin solution in 100% human serum was used as a working stock solution. This working stock solution was serially diluted in pooled normal human serum starting with a 4-fold dilution factor, using 2-fold serial dilutions, and producing five noggin control solutions with the following noggin concentrations: 18, 9, 4.5, 2.25 and 1.13 μg/mL in 100% serum (300, 150, 75, 37.5 and 18.8 ng/mL final concentration after 1:60 dilution). As shown in Tables 8 and 9, the intra-day precision (%CV) of the CPS signal was within 20% while the inter-day variability of the CPS signal was not greater than 25%.

TABLE 8
Intra-Day Variability of Noggin Positive Control Solutions

| | 300 | 150 | 75 | 37.5 | 18.8 |
|---|---|---|---|---|---|
| | ng/mL | ng/mL | ng/mL | ng/mL | ng/mL |
| Plate ID | Noggin | Noggin | Noggin | Noggin | Noggin |
| 031004_2 | 29129 | 37796 | 38288 | 38492 | 43217 |
| | 30661 | 35828 | 40314 | 40064 | 42771 |
| 031004_3 | 27615 | 38943 | 37544 | 40581 | 41493 |
| | 28258 | 35721 | 38412 | 39685 | 41094 |
| 031004_4 | 29191 | 37543 | 38615 | 39863 | 43007 |
| | 29513 | 36738 | 41933 | 42916 | 41624 |
| Mean | 29061 | 37095 | 39184 | 40267 | 42201 |
| Std. Dev. | 1051 | 1243 | 1628 | 1470 | 902 |
| % CV | 3.6 | 3.4 | 4.2 | 3.7 | 2.1 |
| 031018_1 | 17457 | 25608 | 32309 | 41172 | 37556 |
| | 18295 | 28756 | 37324 | 36585 | 35715 |
| 031018_3 | 17933 | 28100 | 34257 | 34536 | 30232 |
| | 20396 | 23867 | 28407 | 38687 | 36371 |
| 031022_1 | 20003 | 22562 | 32069 | 33276 | 34856 |
| | 20950 | 19346 | 29773 | 31087 | 34571 |
| 031022_3 | 25446 | 25186 | 42453 | 44303 | 45416 |
| | 25454 | 24664 | 38963 | 42040 | 47265 |
| Mean | 20741 | 24761 | 34444 | 37711 | 37747 |
| Std. Dev. | 3156 | 2999 | 4801 | 4630 | 5737 |
| % CV | 15.2 | 12.1 | 13.9 | 12.3 | 15.2 |

TABLE 9
Inter-Day Variability of Noggin Positive Control Solutions

| | 300 | 150 | 75 | 37.5 | 18.8 |
|---|---|---|---|---|---|
| | ng/mL | ng/mL | ng/mL | ng/mL | ng/mL |
| | Noggin | Noggin | Noggin | Noggin | Noggin |
| Mean | 24307 | 30047 | 36475 | 38806 | 39656 |
| Std. Dev. | 4903 | 6750 | 4400 | 3754 | 4823 |
| % CV | 20.2 | 22.5 | 12.1 | 9.7 | 12.2 |

The positive control dilution at which the CPS value is equal to the CPS value of the 100 ng/mL rhBMP-2 standard was determined for these samples by interpolation. The positive control sample at this dilution was assigned a UNA of 50. The final reportable UNA values were calculated for the positive control working stock solution containing 72 μg/mL of noggin in 100% human serum as described above.

As shown in Table 10, the intra-day variability of the final UNA values was within 5% while the inter-day variability was within 10%. The average inter-day UNA value was 78.5. Based on the determined inter-day variability standard deviation value of 5.9 UNA, the 95% confidence interval for the positive control noggin stock solution was determined to be between 64.7 and 92.3 UNA.

TABLE 10
Variability of the UNA Values of Noggin Positive Controls

| | |
|---|---|
| Plate ID | UNA |

| | | Intra-Day Variability | |
|---|---|---|---|
| 031004_2 | 73.1 | Mean | 73.9 |
| | 74.1 | Std. Dev. | 0.7 |
| 031004_3 | 73.2 | % CV | 1.0 |
| | 74.9 | | |
| 031004_4 | 73.8 | | |
| | 74.3 | | |
| 031018_1 | 85.3 | Mean | 81.2 |
| | 80.7 | Std. Dev. | 3.7 |
| 031018_3 | 75.3 | % CV | 4.6 |
| | 83.3 | | |
| 031022_1 | 85.0 | Mean | 88.1 |
| | 95.7 | Std. Dev. | 4.8 |
| 031022_3 | 83.1 | % CV | 5.4 |
| | 88.6 | | |
| 031218_1 | 74.1 | Mean | 76.2 |
| | 77.6 | Std. Dev. | 1.5 |
| | 76.7 | % CV | 2.0 |
| | 74.7 | | |
| | 77.9 | | |
| 031218_4 | 77.2 | Mean | 76.1 |
| | 77.8 | Std. Dev. | 2.8 |
| | 71.2 | % CV | 3.7 |
| | 78.1 | | |
| | | Inter-Day Variability | |
| | | Mean | 78.5 |
| | | Std. Dev. | 5.9 |
| | | % CV | 7.5 |

A solution containing 0 ng/mL of noggin and 0 ng/mL of rhBMP-2 was used in the assay as a blank. The inter-day variability of the blank was assessed in the C36 assay. As shown in Table 11, the overall %CV for the CPS values for this sample was within 15%.

TABLE 11
Inter-Day Variability of CPS Values of Blank Solution

| | |
|---|---|
| Plate ID | CPS Values |

| | | |
|---|---|---|
| 030910_2 | 22279 | 23031 |
| 030910_3 | 23805 | 23326 |
| 030918_1 | 19040 | 17390 |
| 030918_2 | 23923 | 20623 |
| 030918_3 | 18199 | 19072 |
| 030918_4 | 21163 | 20896 |
| 030920_1 | 24391 | 22386 |
| 031004_2 | 25167 | 25516 |
| 031004_3 | 25537 | 24684 |
| 031004_4 | 25358 | 24116 |
| 031018_1 | 23266 | 22258 |
| 031018_3 | 18115 | 18306 |
| 031022_1 | 21559 | 21065 |
| 031022_3 | 26873 | 24813 |
| Mean | 22363 | |
| Std. Dev. | 2652 | |
| % CV | 11.9 | |

### Example 13

Matrix Interference

A group of 32 individual human serum samples was analyzed to evaluate potential matrix interference in the C36 assay. Samples were analyzed with or without spiking with 200 ng/mL of rhBMP-2 as described above. Results are shown in Tables 12 and 13, respectively.

TABLE 12
Matrix Interference - Samples with 200 ng/mL rhBMP-2

| | | | | | CPS of 100 |
|---|---|---|---|---|---|
| | | Raw CPS | Sample | Sample | ng/mL |
| Plate ID | Sample ID | Signal | UNA | Tested As: | rhBMP-2 |
| 030918_4 | 40 | 48918 | <50 | Negative | 38700 |
| | 41 | 47586 | <50 | Negative | |
| | 42 | 47651 | <50 | Negative | |
| | 44 | 47150 | <50 | Negative | |
| | 45 | 43994 | <50 | Negative | |
| | 47 | 50859 | <50 | Negative | |
| | 48 | 48905 | <50 | Negative | |
| | 49 | 44864 | <50 | Negative | |
| 030918_2 | 101 | 61944 | <50 | Negative | 39987 |
| | 103 | 49529 | <50 | Negative | |
| | 105 | 45600 | <50 | Negative | |
| | 108 | 58771 | <50 | Negative | |
| | 110 | 49415 | <50 | Negative | |
| | 116 | 47998 | <50 | Negative | |
| | 117 | 48623 | <50 | Negative | |
| | 118 | 45870 | <50 | Negative | |
| 030918_3 | 129 | 46135 | <50 | Negative | 37813 |
| | 131 | 46990 | <50 | Negative | |
| | 133 | 49638 | <50 | Negative | |
| | 134 | 46145 | <50 | Negative | |
| | 136 | 47093 | <50 | Negative | |
| | 137 | 50054 | <50 | Negative | |
| | 138 | 48668 | <50 | Negative | |
| | 018 | 45935 | <50 | Negative | |
| 030918_1 | 211 | 41691 | <50 | Negative | 28787 |
| | 212 | 38562 | <50 | Negative | |
| | 213 | 41920 | <50 | Negative | |
| | 216 | 43032 | <50 | Negative | |
| | 217 | 38972 | <50 | Negative | |
| | 218 | 43190 | <50 | Negative | |
| | 219 | 42413 | <50 | Negative | |
| | 220 | 39238 | <50 | Negative | |
| Mean | | 46792 | | | |
| Std. Dev. | | 4883 | | | |
| % CV | | 10.4 | | | |

TABLE 13
Matrix Interference - Samples without rhBMP-2

| | | | | | CPS of 100 |
|---|---|---|---|---|---|
| | | Raw CPS | Sample | Sample | ng/mL |
| Plate ID | Sample ID | Signal | UNA | Tested As: | rhBMP-2 |
| 030918_4 | 40 | 20943 | >50 | Positive | 38700 |
| | 41 | 22197 | >50 | Positive | |
| | 42 | 21945 | >50 | Positive | |
| | 44 | 20327 | >50 | Positive | |
| | 45 | 20602 | >50 | Positive | |
| | 47 | 24910 | >50 | Positive | |
| | 48 | 22025 | >50 | Positive | |
| | 49 | 22141 | >50 | Positive | |
| 030918_2 | 101 | 28466 | >50 | Positive | 39987 |
| | 103 | 24100 | >50 | Positive | |
| | 105 | 25791 | >50 | Positive | |
| | 108 | 27046 | >50 | Positive | |
| | 110 | 28984 | >50 | Positive | |
| | 116 | 23412 | >50 | Positive | |
| | 117 | 27668 | >50 | Positive | |
| | 118 | 24021 | >50 | Positive | |
| 030918_3 | 129 | 20590 | >50 | Positive | 37813 |
| | 131 | 22263 | >50 | Positive | |
| | 133 | 19377 | >50 | Positive | |
| | 134 | 22015 | >50 | Positive | |
| | 136 | 18997 | >50 | Positive | |
| | 137 | 21927 | >50 | Positive | |
| | 138 | 22628 | >50 | Positive | |
| | 018 | 20532 | >50 | Positive | |
| 030918_1 | 211 | 18508 | >50 | Positive | 28787 |
| | 212 | 18037 | >50 | Positive | |
| | 213 | 17066 | >50 | Positive | |
| | 216 | 18537 | >50 | Positive | |
| | 217 | 18261 | >50 | Positive | |
| | 218 | 20251 | >50 | Positive | |
| | 219 | 21000 | >50 | Positive | |
| | 220 | 19369 | >50 | Positive | |
| Mean | | 21998 | | | |
| Std. Dev. | | 3096 | | | |
| % CV | | 14.1 | | | |

When samples were analyzed with the addition of the 200 ng/mL of rhBMP-2, all individual sera samples generated CPS values above that for the 100 ng/mL rhBMP-2 standard analyzed on the same plate. Samples therefore were assigned UNA values of less than 50 (scored negative).

As expected, samples analyzed without added rhBMP-2 generated CPS values below that for the corresponding 100 ng/mL rhBMP-2 standard and were assigned UNA values greater than 50 (scored as apparent positives). This experiment demonstrated the lack of matrix components that could potentially interfere with the BMP-2/ luciferase signaling pathway in these cells. In this test, no evidence of matrix interference was found.

All references cited herein are incorporated herein by reference in their entirety and for all purposes to the same extent as if each individual publication or patent or patent application was specifically and individually indicated to be incorporated by reference in its entirety for all purposes. To the extent publications and patents or patent applications incorporated by reference contradict the disclosure contained in the specification, the specification is intended to supercede and/or take precedence over any such contradictory material.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Many modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with a true scope and spirit of the invention being indicated by the following claims.

We claim:

1. An assay for the detection of neutralizing antibodies to a bone morphogenetic protein (BMP) comprising:
(a) incubating an antibody to be evaluated with a BMP;
(b) incubating the product of step (a) with BMP-responsive cells containing a BMP-responsive reporter construct,
wherein the antibody of step (a) is neutralizing if the level of expression of the reporter gene in the cells of step (b) is less than the level of expression of the reporter gene in the same cells incubated with BMP in the absence of the antibody.

2. The assay of claim 1 wherein the BMP is BMP-2.

3. The assay of claim 1 wherein the BMP is rhBMP-2.

4. The assay of claim 1 wherein the cells are C36 cells.

5. The assay of claim 1, wherein the reporter construct comprises a Msx-2 promoter.

6. The assay of claim 1, wherein the reporter construct comprises an ID-1 promoter.

7. The assay of claim 5, wherein the reporter construct comprises a luciferase gene.

8. The assay of claim 1, wherein the antibody is in a serum sample.

9. The assay of claim 1, wherein the antibody is in a human serum sample.

10. The assay of claim 1, wherein the antibody is in a heat inactivated and delipidated serum sample.

11. The assay of claim 1, further comprising a positive control capable of neutralizing at least one biological activity of the BMP.

12. The assay of claim 11, wherein the positive control is an antibody specific for the BMP.

13. The assay of claim 12, wherein the antibody is a monoclonal antibody.

14. The assay of claim 12, wherein the antibody is a polyclonal antibody.

15. The assay of claim 12, wherein the antibody is specific for rhBMP-2.

16. The assay of claim 15, wherein the antibody is monoclonal antibody MAB3552.

17. The assay of claim 11, wherein the positive control is a non-antibody molecule capable of binding to the BMP and neutralizing at least one biological activity of the BMP.

18. The assay of claim 17, wherein the positive control is a non-antibody molecule capable of binding to rhBMP-2 and neutralizing at least one biological activity of rhBMP-2.

19. The assay of claim 18, wherein the positive control is chordin.

20. The assay of claim 18, wherein the positive control is noggin.

21. A rhBMP-2 responsive cell comprising a DNA molecule comprising a Msx-2 promoter operably linked to a luciferase reporter gene so that the Msx-2 promoter controls expression of the luciferase gene.

22. The rhBMP-2 responsive cell of claim 21, wherein the cell is a C36 cell.

23. A rhBMP-2 responsive cell comprising a DNA molecule comprising a ID-1 promoter operably linked to a luciferase reporter gene so that the Msx-2 promoter controls expression of the luciferase gene.

24. The rhBMP-2 responsive cell of claim 23, wherein the cell is a C36 cell.

25. An assay for the detection of neutralizing antibodies to a BMP comprising the step of incubating an antibody, a BMP, and BMP-responsive cells containing a BMP-responsive reporter construct, wherein the antibody is neutralizing if the resulting level of expression of the reporter gene is less than expression of the reporter gene in the same cells incubated with BMP alone.

26. The assay of claim 25, wherein the sample, BMP, and cells are combined at the same time.

27. The assay of claim 25, wherein the BMP and cells are combined before the sample is added.

28. The assay of claim 25, wherein the expression of the reporter gene is measured prior to adding the sample and wherein this serves as a negative control.
